(19) United States
(12) Patent Application Publication
(10) Pub. No.: US 2024/0016456 A1
(43) Pub. Date: Jan. 18, 2024

(54) LONGITUDINAL DATASETS AND MACHINE LEARNING MODELS FOR MENOPAUSE STATE AND ANOMALY PREDICTIONS

(71) Applicant: SRI International, Menlo Park, CA (US)

(72) Inventors: Massimiliano de Zambotti, Sunnyvale, CA (US); Fiona Baker, San Jose, CA (US); Andreas Tsiartas, San Jose, CA (US)

(73) Assignee: SRI International, Menlo Park, CA (US)

(21) Appl. No.: 18/034,464

(22) PCT Filed: Nov. 3, 2021
(86) PCT No.: PCT/US2021/057869

§ 371 Date: Apr. 28, 2023

Related U.S. Application Data
(60) Provisional application No. 63/108,987, filed on Nov. 3, 2020.

(51) Int. Cl. A61B 5/00; A61B 5/0205

(52) U.S. Cl. CPC A61B 5/7275; A61B 5/7264; A61B 5/02055

(57) ABSTRACT

Embodiments are directed to a non-transitory computer-readable storage medium comprising instructions that when executed cause processor circuitry to generate a longitudinal dataset of a set of features from tracked physical measurements of a user received from sensor circuitry, apply at least one machine learning model to the longitudinal dataset of the set of features to identify a pattern within the set of features indicative of a probability of the user being in a state of menopause, predict a current state of menopause for the user based on the identified pattern, and communicate a data message indicative of the current state of menopause for the user. In some embodiments, the set of features are compressed to pseudo-features and input to a first ML model using a second ML model.

### BACKGROUND

Menopause is a point in time after a biological female stops having menstrual periods, generally referred to as the point in time twelve months after the last period. During the transition time, many women experience hot flashes, trouble sleeping, pain during sex, moodiness, irritability, and/or depression, among other symptoms. There are different states of menopause including menopause transition, sometimes referred to as “perimenopause”, and post-menopause states. During the menopause transition state, hormone levels are changing and menstrual period cycles can become irregular, but still occur. For example, the ovaries may reduce producing estrogen and other sex hormones during the menopause transition state. At the point of transition, the ovaries no longer release eggs and estrogen production falls below a threshold level. While there are general trends for age and timing of the different menopause states, as well as symptoms of the states, particular females can experience menopause differently and which may be difficult to identify due to a cross-range of menopause features. Many women desire information on user specific menopause states for different purposes, such as fertility or family planning and for properly treating menopause symptoms.

### SUMMARY

The present invention is directed to overcoming the above-mentioned challenges and others related to tracking menopause, such as those involving predicting a menopause state and/or menopause-related anomaly using longitudinal datasets of tracked features.

Various embodiments of the present disclosure are directed to a non-transitory computer-readable storage medium comprising instructions that when executed cause processor circuitry to generate a longitudinal dataset of a set of features from tracked physical measurements of a user received from sensor circuitry, apply at least one machine learning model to the longitudinal dataset of the set of features to identify a pattern within the set of features indicative of a probability of the user being in a state of menopause, predict a current state of menopause for the user based on the identified pattern, and communicate a data message indicative of the current state of menopause for the user.

In some embodiments, the non-transitory computer-readable storage medium further includes instructions executable to communicate the data message indicative of the current state of menopause in response to the current state being different than a past predicted state of menopause for the user.

In some embodiments, the at least one machine learning model includes at least a first machine learning model applied to identify the pattern within the set of features indicative of the probability of the user being in the state of menopause and a second machine learning model, and further including instructions executable to apply the second machine learning model to the set of features to identify a menopause-related anomaly for the user and in the set of features, wherein each of the first machine learning model and the second machine learning model include a plurality of different patterns of the set of features which are indicative of different states of menopause, including the state and the pattern.

In some embodiments, the second machine learning model is used to generate pseudo-features from the set of features and to identify the menopause related anomaly from an output of the second machine learning model, wherein the output of the second machine learning model includes an indicator of the menopause-related anomaly associated with a divergence from at least one of: a baseline pattern of the set of features for the user and general population trends.

In some embodiments, the physical measurements include at least two or more sensor signals indicative of: heart rate, skin temperature, skin conductance, motion, pH levels, moisture, environmental temperature, environmental humidity, photoplethysmogram (PPG), and combinations thereof. In some embodiments, the physical measurements include each of heart rate, skin temperature, skin conductance, motion, pH levels, moisture, environmental temperature, environmental humidity, and PPG.

In some embodiments, the set of features include features selected from: menstrual cycle, changes in vaginal characteristics, changes in skin characteristic, hot flash events, sleep disturbances, autonomic nervous system function, heart rate variability, temperature, and combinations thereof.

In some embodiments, the non-transitory computer-readable storage medium further includes instructions executable to: train the at least one machine learning model using input data including general population trends of features and demographic information associated with the user and expected outputs of the at least one machine learning model including at least one indicator of a menopause state and a menopause-related anomaly; revise the at least one machine learning model for the user based on a baseline pattern of the set of features for the user identified using at least a portion of the longitudinal dataset of the set of features; and identify the pattern within the longitudinal dataset of the set of features indicative of the probability of the user being in the state of menopause based on a change from the baseline pattern of the set of features and the general population trends.

In some embodiments, the at least one machine learning model and the identified pattern include a plurality of sub-models using to generate a plurality of sub-patterns for different subsets of features of the set of features, and further including instructions executable to apply the at least one machine learning model to the longitudinal dataset to obtain a confidence score for each of the plurality of sub-models and predict the current state of menopause based on the confidence score for each of the plurality of sub-models. In some embodiments, each of the confidence scores are weighted based on at least one of: a level of predictiveness of the subset of features and a reliability of sensor signals associated with the subset of features.

In some embodiments, the non-transitory computer-readable storage medium further includes instructions executable to align features of the set of features to a common time point and to predict the current state of menopause for the user based on a plurality of past predicted states of menopause for the user.

Various embodiments are directed to a non-transitory computer-readable storage medium comprising instructions that when executed cause processor circuitry of a computing device to generate a longitudinal dataset of a set of features from tracked physical measurements of a user received from sensor circuitry, identify a baseline pattern of the set of features for the user using at least a portion of the longitudinal dataset of the set of features, generate at least one machine learning model based on the baseline pattern of the set of features and general population trends of a demographic population associated with the user, identify a menopause-related anomaly in the set of features using the at least one machine learning model and based on a change from the baseline pattern of the set of features and the general population trends, and communicate a data message indicative of the anomaly to the user.

In some embodiments, the instructions to communicate the data message include instruction executable to automatically send the data message to the user indicative of an issue associated with the menopause-related anomaly and notify the user to contact a professional, and wherein the menopause-related anomaly includes an abnormal transition between menopause states.

In some embodiments, the at least one machine learning model includes a first machine learning model indicative of a prediction of a current state of menopause for the user and a second machine learning model applied to generate pseudo-features from the set of features and identify the menopause-related anomaly, and further including instructions executable to predict the current state of menopause for the user using the first machine learning model that identifies the pattern within at least one of the pseudo-features and the menopause-related anomaly indicative of a probability of the user being in a state of menopause, wherein the communicated data message is indicative of the menopause-related anomaly and the current state of menopause.

In some embodiments, each of the first machine learning model and the second machine learning data model include a plurality of sub-models for different groups of features of the set of features, and the instructions to apply the first machine learning model to the longitudinal data set include instructions executable to obtain a confidence score for each of the plurality of sub-models and to predict the current state of menopause based on the confidence score for each of the plurality of sub-models.

Various embodiments are directed to a system, comprising sensor circuitry, including communication circuitry, configured to obtain sensor signals indicative of physical measurements associated with a user and to communicate the physical measurements, and processor circuitry. The processor circuitry is configured to generate a longitudinal dataset of a set of features from the physical measurements of the user as tracked over time, identify a baseline pattern of the set of features for the user identified using at least a portion of the longitudinal dataset of the set of features, generate at least one machine learning model based on the baseline pattern of the set of features and general population trends of a demographic population associated with the user, identify a menopause-related anomaly in the set of features using the at least one machine learning model and based on a change from the baseline pattern of the set of features and the general population trends, identify a pattern within the set of features indicative of a probability of the user being in a state of menopause using the at least one machine learning model and at least one of the set of features and the menopause-related anomaly, and communicate a data message indicative of the menopause-related anomaly to the user.

In some embodiments, the processor circuitry is configured to identify the anomaly in the set of features from the pattern using the at least one machine learning model and based on a deviation from the baseline pattern and general population trends of the demographic population associated with the user.

In some embodiments, the at least one machine learning model includes a first machine learning model indicative of the probability of the user being in the state of menopause and a second machine learning model that includes an encoder/decoder pair applied to generate pseudo-features from the set of features and to identify the menopause related anomaly from a reconstruction error, and the processor circuitry is configured to apply the first machine learning model to the pseudo-features to predict a current state of menopause for the user based on the identified pattern within the set of features indicative of the probability of the user being in the state of menopause, wherein the current state of menopause is associated with a transition to or from a state selected from the group consisting of: pre-menopause, menopause transition, and post-menopause.

In some embodiments, the processor circuitry is configured to predict the current state of menopause for the user based on at least two or more of: the identified pattern, a plurality of past predicted states of menopause for the user, the pseudo-features, and the menopause-related anomaly. In some embodiments, the processor circuitry is to predict the current state of menopause for the user based one each of the identified pattern, a plurality of past predicted states of menopause for the user, the pseudo-features, and the menopause-related anomaly, or various combinations thereof.

In some embodiments, the system further includes input circuitry configured to receive information from the user, wherein the set of features include the received information.

In some embodiments, the processor circuitry is configured to identify the menopause-related anomaly in the set of features using the at least one machine learning model that includes a neural network data model with hidden states to identify the baseline pattern and the menopause-related anomaly is identified from the baseline pattern using the longitudinal data set, wherein the baseline pattern includes a plurality of sub-patterns associated with the set of features.

### BRIEF DESCRIPTION OF THE DRAWINGS

Various example embodiments can be more completely understood in consideration of the following detailed description in connection with the accompanying drawings, in which:

FIG. 1 illustrates an example computing device including non-transitory computer-readable medium storing executable instructions, in accordance with the present disclosure.

FIG. 2 illustrates an example computing device including non-transitory computer-readable medium storing executable instructions, in accordance with the present disclosure.

FIG. 3 illustrates an example system including sensor circuitry and processor circuitry, in accordance with the present disclosure.

FIG. 4 illustrates an example multi-modal bio-behavioral tracking system, in accordance with the present disclosure.

FIG. 5 illustrates an example autoencoder for memorizing patterns of input features using longitudinal data, in accordance with the present disclosure.

FIG. 6 illustrates an example of a predictive data model, in accordance with various embodiments.

FIG. 7 illustrates an example system for predicting menopause states and identifying menopause-related anomalies, in accordance with various embodiments.

### DETAILED DESCRIPTION

In the following detailed description, reference is made to the accompanying drawings which form a part hereof, and in which is shown by way of illustration specific examples in which the disclosure can be practiced. It is to be understood that other examples can be utilized, and various changes may be made without departing from the scope of the disclosure. The following detailed description, therefore, is not to be taken in a limiting sense, and the scope of the disclosure is defined by the appended claims. It is to be understood that features of the various examples described herein may be combined, in part or whole, with each other, unless specifically noted otherwise.

The evolution of smart devices to track multi-modal bio-behavioral feature tracking (e.g., sleep, physical activity, body temperature, hormones, etc.) powered by advancements in artificial intelligence (AI) and machine learning (ML) integration, has opened new opportunities in health care and wellness sectors, rendering medical and health information accessible to consumers. For example, the use of wearable technology in the field of women's health is on the rise, such as for purposes of menstrual cycle tracking for fertility windows prediction. Longitudinal data tracking and personalized approaches can be used to precisely measure and understand psychophysiology, recognize anomalies, and monitor overall health of an individual.

A critical natural stage in a woman's life is menopause, which is defined as the absence of spontaneous menstrual bleeding for more than twelve months. Menopause is sometimes considered a moment of transition (e.g., the last period), and not a state. Natural menopause occurs at about 51 years of age (±10 years). However, the menopause transition (e.g., the years leading up to menopause, sometimes referred to as “perimenopause”) begins four to six years (or more) before the cessation of menses, and is accompanied by several psychophysiological changes including fluctuating hormone levels (e.g., toward progressive slowing in ovarian estrogen production), irregular periods, sleep disturbances, emotional changes, vaginal dryness, urinary urgency, weight gain, and the occurrence of other menopause-unique symptoms, such hot flashes. These complex changes show feature-specific and woman-specific temporal dynamics and manifestations, and can interfere with daily activities, impacting health and well-being for a user. Further, many women have difficulty recognizing they are entering the menopause transition or experiencing symptoms that may be related to the approach of menopause. Not only is there variability in the symptoms and changes a woman may experience, there is also inter-individual variability in the length of time of the menopausal transition leading up to menopause. Due to the inter-individual variability, women are often unaware that they are experiencing menopause transition and/or an abnormal menopause transition. Embodiments in accordance with the present disclosure are directed to tracking a set of features for a user (e.g., woman) over time to generate a longitudinal dataset of features, specific to the user, and using the longitudinal dataset and at least one ML model to predict a menopause status and/or identify anomalies in the approach to menopause and/or the menopause transition.

Various different features can be considered for menopause patterns and predicting the status of menopause for a particular user. Age at the menopause transition is an important predictor of female health given that both early and late menopause are associated with several adverse consequences including an increased risk for breast and ovarian cancer, cardiovascular disease, osteoporosis, and cognitive decline. Predicting the onset of menopause can involve considering several features including age and menstrual cycle status, which alone do not give an accurate individualized prediction of menopause occurrence/status. In addition, sporadic sampling of follicle-stimulating hormone (FSH), used in the classification of reproductive ageing, shows weak prediction capability, possibly due to its high variability in levels across the menstrual cycle and fluctuations at different times. Better prediction of age at menopause can be made by considering the levels of anti-müllerian hormone (AMH), a reproducible, stable and reliable feature that can reflect changes in the follicular pool, however, frequent sampling of any hormone with current technology is challenging to achieve in non-clinical or non-laboratory settings. The age of the user's mother at menopause transition can offer another predicting factor, underlying the degree of heritability of menopause.

The age of menopause transition can be used for other purposes. For example, age at menopause and anomalies in the menopause transition can result in future health issues. Predicting age at menopause and mapping of normality and anomalies during the menopause transition is a unique window of opportunity for health risk mitigation.

Embodiments in accordance with the present disclosure involving predicting or recognizing menopause states and menopause-related anomalies that can be applied outside the clinic by using sensor circuitry worn by the user to track the set of features over time, and in a manner that is not intrusive to the user. For example, the system can track multi-modal biological changes in women approaching menopause via wearable biosensors data assessment; extracting, processing and integrating menopause-relevant bio-features; assessing/predicting a woman's menopausal state and deviations from normally occurring menopause via advanced AI analytics. The system can monitor and characterize menopausal status and anomalies via continuous multimodal wearable biosensors data assessment, integration and analysis. Electronic momentary assessments (e.g., self-reported menstrual tracking) can complement sensor signals gathered and the feature extraction. “State of menopause” and “menopause state” are used herein interchangeably.

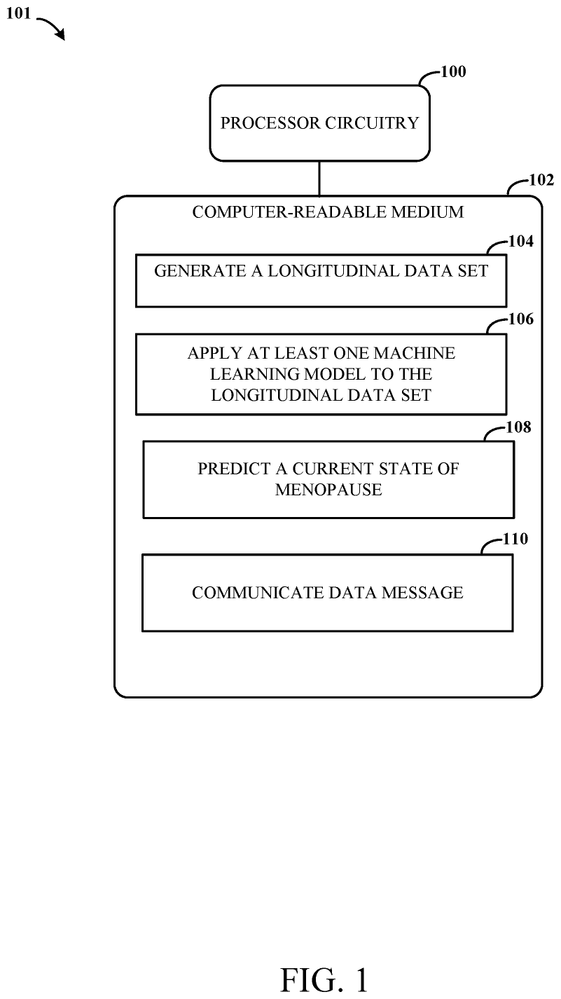

Turning now to the figures, FIG. 1 illustrates an example computing device including non-transitory computer-readable medium storing executable instructions, in accordance with the present disclosure.

In some embodiments, the processor circuitry 100 can form part of a computing device 101. The computing device 101 includes the processor circuitry 100 and computer-readable medium 102 storing a set of instructions 104, 106, 108, 110. Although embodiments are not so limited, and the processor circuitry 100 and computer-readable medium 102 can form a part of distributed computing devices, with the distributed computing devices being in communication with each other. The computer-readable medium 102 can, for example, include read-only memory (ROM), random-access memory (RAM), electrically erasable programmable read-only memory (EEPROM), Flash memory, a solid state drive, and/or discrete data register sets. As used herein, “executable instructions” can be interchanged with “instructions executed by the processor circuitry”.

At 104, the processor circuitry 100 can execute instructions to generate a longitudinal dataset of a set of features from tracked physical measurements associated with a user (e.g., a female) as received from sensor circuitry. A longitudinal dataset, as used herein, includes and/or refers to data associated with the same type of information and on the same user over time, such as tracked sensor signals indicative of physical measurements of a particular user over multiple months and/or years. The sensor signals and/or physical measurements can include physiological and/or bio-behavioral measurements, such as heart rate (HR), blood pressure, skin temperature, skin conductance (SC), body motion (e.g., from accelerometer and/or global positioning data (GPS), hormone levels (e.g., in interstitial fluid through use of a skin patch), vaginal characteristics (e.g., pH levels or moisture levels), skin characteristics (e.g., skin dryness via moisture sensor), and photoplethysmograph (PPG), among others measures as well as different combinations thereof. The physical measurements can further include environmental measurements, such as air temperature and humidity. Sensor signals obtained by the sensor circuitry can be indicative of the physical measurements and can be communicated to the processor circuitry 100.

In some embodiments, the sensor circuitry can obtain the physical measurements, such as physiological, bio-behavioral, and/or environmental measurements. The sensor circuitry can include wearable technology, such as wearable sensors, implantable sensors, and/or environmental sensors, among other types of sensors. Wearable technology, as used herein, includes and/or refers to one or more sensors, and/or device with at least one sensor that is wearable and used to obtain physical (e.g., physiological) measurements from the user. In some embodiments, the wearable technology can be continuously worn by the user for a period of time or periods of time (e.g., for a day, for multiple days, for months, all day, all night). Non-limiting examples of wearable technology include a smart watch, fitness watch, and/or smart ring, among others.

The set of features can be obtained by processing the sensor signals received from the sensor circuitry. The set of features can include menstrual cycle, changes in vaginal characteristics (e.g., pH levels, dryness), changes in skin characteristics (e.g., drying and/or aging), hot flash events, sleep disturbances, autonomic nervous system (ANS) function, heart rate variability, temperature, and combinations thereof. As noted above, the features can be physiological and bio-behavioral. In some embodiments, the processor circuitry 100 can generate the longitudinal dataset of the set of features by processing the sensor signals over time to track the physical measurements, including changes in patterns of the physical measurements.

In some embodiments, the user can input self-reported measures to the computing device 101. The self-reported measures can complement physical measurement tracking. The self-reported measures can include menstrual cycle irregularities, changes in vaginal pH levels, vaginal dryness, skin aging, hot flashes, night sweats, sleep disturbances, ANS function, among others. In some embodiments, the self-reported measures are used to supplement the physical measures (e.g., physiological) and/or as feedback data. For example, the processor circuitry 100 can use the physical measures and the self-reported measures to generate the longitudinal dataset of the set of features and which is used to provide multi-modal bio-behavioral tracking for menopause states and/or anomalies. In some embodiments or in addition, the self-reported measures can be used as feedback data to confirm or deny the predicted current menopause state and/or identified menopause-related anomaly. The multi-modal bio-behavior tracked can include patterns of sleep, physical activity, body temperature, menstrual cycles, hormones, among other user behaviors and cycles, as well as changes therein.

The physical measurements obtained from the sensor signals (or data) and/or the self-reported measures can include and/or be associated with menstrual cycles, such as menstrual cycle irregularities. Menopause can be characterized by menstrual cycle irregularities, reflecting alteration in the normally occurring coordinated interaction between the hypothalamus, the pituitary, and the ovaries. Several characteristics of a woman's menstrual cycle can be captured (e.g., length, variability, etc.), as further described below.

In some embodiments, menstrual cycles of the user can be tracked over time (e.g., multiple months and/or years) to detect changes in regularity and characteristics, and determine ovulation, via wearable technology (e.g., using body temperature, PPG-derived heart rate and heart rate variability, activity) and considering time of day or state for greater accuracy of measurement, such as during sleep; and site measurements of temperature (skin, vaginal, tympanic), or factors such as multiple skin sites potentially being more accurate than a single site in the face of environmental variability. For example, with continuous nocturnal temperature measurements, menstrual cycles of a user can be tracked by changes in amplitude of the temperature signal, with a reduction in amplitude being a feature of the luteal phase. Self-tracking (e.g., mobile application) of menstrual cycle characteristics can complement sensor-derived data. In some embodiments, potential confounders can be considered, such as the use of hormonal contraceptives (e.g., birth control pills).

In some embodiments, the physical measurements obtained from the sensor signals (sometimes herein interchangeably referred to as “sensor data”) and/or the self-reported measures can include and/or be associated with changes in vaginal pH levels. The changes in vaginal pH levels can be used as a surrogate measure of the gold-standard Follicle-Stimulating Hormone (FSH) level. In some embodiments, the vaginal pH levels can be measured via wearable technology and/or via self-reporting. Potential confounders can be considered, such as vaginitis.

In some embodiments, the physical measurements obtained from the sensor signals and/or the self-reported measures can include and/or be associated with vaginal dryness. Vaginal dryness is a common symptom of menopause. In some embodiments, vaginal dryness can be measured via wearable technology and/or via self-reporting.

In some embodiments, the physical measurements obtained from the sensor signals and/or the self-reported measures can include and/or be associated with skin aging (e.g., dry skin, skin pH level). For example, skin aging can be particularly relevant to the post-menopause state of menopause, as further described herein. In some embodiments, the skin aging can be measured via a smart cutaneous wearable technology (e.g., device and/or sensor) and/or via user self-reporting.

In some embodiments, the physical measurements obtained from the sensor signals and/or the self-reported measures can include and/or be associated with hot flashes and/or night sweats. Hot flashes are a symptom among women in a menopause transition (e.g., perimenopause) state of menopause, as further described herein. In some embodiments, the hot flashes can be measured via wearable technology (e.g., skin conductance and other sensors) and/or via self-reporting. In some embodiments, potential confounders can be considered, such as motion, physical activity, external temperature, and alcohol consumption. In some embodiments, hot flash triggers can be measured via wearable technology and/or via self-reporting.

In some embodiments, the physical measurements obtained from the sensor signals and/or the self-reported measures can include and/or be associated with sleep disturbances. Sleep patterns can change with aging and menopause states. In some embodiments, sleep disturbances can be measured via wearable technology and/or via self-reporting.

In some embodiments, the physical measurements obtained from the sensor signals and/or the self-reported measures can include and/or be associated with ANS function. ANS function can change with aging and menopause states. In some embodiments, ANS function can be measured via wearable technology and/or via self-reporting. Potential confounders to consider can include motion, physical activity, and menstrual cycle phase. Another physical feature may be menses flow amount.

In some embodiments, the processor circuitry 100 can implement multi-rate feature processing. Multi-rate feature processing can refer to and/or include processing features that are captured over different sample time periods (e.g., time window) and that involve diversity in types of features. For example, the features can include both physiological features and bio-behavioral features. Physiological features include and/or refer to features related to physiology or to functioning of the body, such as heart rate, skin conductance, and hormone levels. Bio-behavioral features include and/or refer to features related to biological-related behavior of the user, such as times associated with or related to sleep states or transitions (e.g., bed time or wake-up time of the user), motion at or around sleep transitions (e.g., motion when the user wakes up or motion prior to sleep on-set), dietary or other habits (e.g., exercise or medicine), among other bio-cycle, psychological, and/or behavior features. For example, at least some of the bio-behavioral features can be obtained using physiological features, such as determining wake-up or sleep-onset using heart rate and motion measures. To process the set of features, the features can be divided into physiological features and bio-behavioral features. Physiological and bio-behavioral features are complementary and diverse. Thus, the features improve accuracy and robustness to noisy measurements. Such features have collection diversity and feature diversity. Collection diversity refers to and/or includes features that are sampled over different time periods. For example, the features can be: (a) sampled at different days, weeks or years; (b) are not synchronously sampled; and (c) missing samples. Individual features measurements can be contradicting. The information can be captured when modeled jointly. Feature diversity refers to and/or includes the different types of features collected. Multiple features implementation is expensive. Some features can contain or be dependent on demographic information. Others contain user specific information, others both. Diverse features pose implementation and integration challenges, as further described herein.

At 106, the processor circuitry 100 can execute the instructions to apply at least one ML model to the longitudinal dataset to identify a pattern within the set of features indicative of a probability of the user being in a state of menopause. The at least one ML model (e.g., each of the first ML model and second ML model) can include different patterns of features which are indicative of a probability of the user being in the state of menopause. In some embodiments, the at least one ML model includes a first ML model and a second ML model. The first ML model can be indicative of the probability of the user being in the state of menopause and used to predict the current menopause state for the user. For example, the first ML model can be applied to identify the pattern within the set of features indicative of the probability of the user being in the state of menopause. The second ML model can be indicative of patterns of features of the user and/or other users in known and stable menopause states, and which can be used to generate pseudo-features from the set of features and/or identify menopause-related anomalies in the set of features.

Pseudo-features include and/or refer to compressed forms of the set of features, with the number of pseudo-features being less than the number of the set of features (e.g., 100 features are compressed into 5 pseudo-features). The pseudo-features can each include subsets of the set of features, with each pseudo-feature respectively retaining information from each feature in the subset. In some embodiments, as further described herein, the set of features input to a respective ML model can include pseudo-features and/or a reconstruction error obtained using another ML model (e.g., an autoencoder). For example, the second ML model can be used to generate the pseudo-features from the set of features, and the pseudo-features can be input to the first ML model to predict the current menopause state from the user.

The different states of menopause can include pre-menopause, menopause transition, and post-menopause. As used herein, the menopause transition state, sometimes referred to as perimenopause and/or early and late menopause, includes and/or refers to a state in which the user is transitioning (but has not transitioned) to menopause. During the menopause transition state, which is often when a user is in their forties, indications and/or signs (e.g., symptoms) of menopause may be experienced or exhibited, but the user may still be in a reproductively-active state. A reproductively-active state, as used herein, includes and/or refers to a state in which the user is capable of becoming pregnant. The menopause transition can be associated with features and patterns of features, such as irregular periods, hot flashes, sleep problems, mood changes or swings, vaginal and bladder problems, reduction in estrogen levels, reduction in fertility, changes in sexual function, bone loss, and reduction in cholesterol levels. Early menopause (sometimes referred to as “early perimenopause”) can be associated with changes of menstrual cycles of seven days or more in length of the menstrual cycle, whereas late menopause (sometimes referred to as “late perimenopause”) can be associated with a space of sixty days or more (e.g., sixty days, ninety days) between periods, among other differences. The menopause transition can occur over a period of years, such as four or six to ten years. Pre-menopause includes and/or refers to the state which is prior to the menopause transition, and the user is in the reproductively-active state (which can be referred to as the normal reproductive state for the user). During pre-menopause, indications and/or signs of menopause may not be experienced and/or exhibited.

The features and/or patterns of features for the menopause transition state (e.g., the changes or reductions in different features) can be a change or reduction with respect to the features and/or patterns during the pre-menopause state. For example and as further described herein, baseline patterns for a user can be identified while the user is in the pre-menopause state. However, embodiments are not so limited and baseline patterns can be identified for different menopause states.

Post-menopause includes and/or refers to a state that is twelve months after the last menstrual period of user. The user may experience and/or exhibit indications and/or symptoms during post-menopause and is in a reproductively-inactive state. A reproductive-inactive state, as used herein, includes and/or refers to a state in which the user is incapable of becoming pregnant and does not experience menstrual periods. Early post-menopause and late post-menopause include different features and patterns, with early post-menopause generally including the first six years from the menopause transition and late post-menopause including ten years and more from the menopause transition. As previously described, the menopause includes and/or refers the point in time of the last menstrual period of the user.

At 108, the processor circuitry 100 can predict a current state of menopause for the user based on the identified pattern(s). The current state can be based on, for example, the menopause state with the greatest probability or other confidence score output by the at least one ML model (e.g., the first ML model). As further described herein, the predicted current state can be based on past predicted states of menopause for the user. The predicted current state of menopause, which is output by the at least one ML model, can include identification of the state itself and/or a numerical value or score indicative of the current state of menopause or a range within the current state, among other outputs.

At 110, the processor circuitry 100 can execute the instructions to communicate a data message indicative of the current state of menopause for the user. The data message can indicate a change in the current state of menopause, a transition between states or to a new state of menopause, and/or a menopause-related anomaly. In some embodiments, the processor circuitry 100 can communicate the data message indicative of the current state of menopause in response to the current state being different than a past predicted state of menopause for the user (e.g., indicative of a transition between states or to the current state). For example, the processor circuitry 100 may not communicate a message if the current state of menopause is predicted to be the same as the past predicted state; however, embodiments are not so limited. As further described below, the data message can be communicated by providing the data message on a display of the computing device 101 and/or communicating to another device for displaying to the user.

In some embodiments, the processor circuitry 100 can generate the at least one ML model. For example, the processor circuitry 100 can generate the at least one ML model based on general population trends and demographic information associated with the user. Generating a ML model, as used herein, can include constructing the ML model and training the constructed ML model using known inputs and known outputs. The ML model can be trained using known inputs that include a set of features for other users, such as demographically similar users (and/or the user), and with the known outputs including indicators of known menopause states (e.g., clinically identified) and/or a menopause-related anomaly, such as the known input features and/or a reconstruction error using the known input features and reconstructed input features (e.g., expected divergence of inputs from reconstructed inputs). The input data used to train the at least one ML model can include external and internal data sources, such as feature sets from other users and/or sensed from the user such that the data models can be demographic (e.g., a representative number of users) or user specific. By providing the known inputs and known outputs, the ML model can be trained.

As described above, the at least one ML model can include a first ML model used to predict a menopause state for the user and a second ML model used to generate pseudo-features from the set of features and identify (e.g., detect) menopause-related anomalies for the user from the set of features. The first ML model can be trained using sets of features from other users and/or expected sets of features based on demographic data as the known inputs and known outputs including indicators of the known menopause states associated with respective sets of features. For example, the first ML model can be trained to identify patterns of features associated with the different menopause states by outputting predicted menopause states based on input feature sets and comparing the predicted menopause states to the known output menopause states to evaluate the performance of the first ML model. Based on the performance, the first ML model can be adjusted (e.g., to achieve the known outputs). In some embodiments, the first ML model can be trained using known inputs that include pseudo-features and menopause-related anomalies, and the above-described known outputs.

The second ML model can be trained using the set of features for other users (and/or the user) and/or expected sets of features for known or stable menopause states. For example, the set of features from the other users (and/or the users) can be used as the known inputs and the known outputs, with the second ML model trained to minimize a reconstruction error rate using the set of features. The second ML model can be trained to learn (e.g., memorize) the different patterns of the set of features and to identify menopause-related anomalies from the learned patterns. In some embodiments, the second ML model is an autoencoder which includes an encoder/decoder pair. The encoder dimensionally compresses the set of features into subsets of features using combinatorial function(s), herein referred to as pseudo-features. Each subset of features is combinatorically combined into a respective pseudo-feature via a function, with the number of pseudo-features being smaller than the number of input features. The decoder reconstructs the input features from the pseudo-features using the functions used to combine the features. The reconstructed input features can be compared to the input features to identify an error respectively between, herein generally referred to as a reconstruction error. In some embodiments, the second ML model is trained by outputting reconstructed input features from the pseudo-features (e.g., as compressed by the encoder and using combinatorial functions) based on input feature sets and comparing the reconstructed input features to evaluate the performance of the model. Based on the performance, the second ML model can be adjusted (e.g., adjust subsets of the set of features that are compressed and/or combinatorial functions). For example, the combinatorial functions (e.g., the functions, weights, features combined) can be adjusted to revise and minimize the reconstruction error using the input features (e.g., a training data set). The threshold can be depend on the number of features and/or type of features (e.g., variability within a feature), and/or be based on the training (e.g., a minimize threshold achieved). The menopause-related anomaly can be identified and/or detected in response to the reconstructed input features having a reconstructed error from the input features that is greater than a threshold, as further described herein As further described herein, in some embodiments, the pseudo-features and/or the reconstruction error can be used as the set of features input to the first ML model. However, embodiments are not so limited, and the second ML model is not limited to an autoencoder and can include other types of ML models.

In some embodiments, the ML models are initially trained using demographic data and/or data of other users such that the ML models can be referred to as a demographic ML model, which may not be specific to the user and/or may be a function of age. The demographic ML models can be based on average trends of patterns of feature sets for a user of the particular demographic (e.g., age, race, hereditary information). The processor circuitry 100 can revise the at least one demographic ML model to be specific to the user to generate a user-specific ML model based on a baseline pattern of the set of features for the user identified using at least a portion of the longitudinal dataset of the set of features. This can include retraining or revising the at least one ML model (e.g., the demographic ML model) using the at least portion of the longitudinal dataset of the set of features. In some embodiments, the portion can include features obtained at a different point in time and/or that are associated with the pre-menopause state for the user. However, embodiments are not so limited and can include baseline patterns for other menopause states. For example, the particular user can exhibit irregular menstrual period cycles while in pre-menopause state and/or have other feature patterns which fall outside the general population trends for patterns of feature sets (e.g., higher heart rate, abnormal menstrual cycle). By establishing the baseline for the user, which can be outside the general population trends, the processor circuitry 100 can better predict current menopause states and transitions as compared to using the demographic predictive data model, as well as identify menopause-related anomalies. For example, the processor circuitry 100 can identify the pattern within the longitudinal dataset indicative of the probability of the user being in the state of menopause based on a change from the baseline pattern of the set of features and the general population trends.

Example demographic patterns of feature sets for a user can be based on age, hormone levels, and/or hotflashes, although embodiments are not so limited. In some embodiments, further demographic patterns of feature sets can be learned by the at least one ML model and/or at least one ML system over time, such as based on sensor signals from a plurality of users. As some examples, the medium duration of menopause transition may be a total 7 years, with medium age at menopause of 52.5 years (in US), and with the average age of menopause transition being around 46-47 years. The averages can be impacted by race or ethnicity, genetics (e.g., when the user's mother transitioned) and lifestyle factors (e.g., smoking, weight, consumption patterns). For example, the transition may start around 45 years to 50 years, may occur for six to ten years, and menopause may occur at the age of around 51-53. In some embodiments, the demographic patterns may be input as feature sets to train the at least one data model, such as patterns as described by E. B. Gold, et al., “Factors Related to Age at Natural Menopause: Longitudinal Analyses from Swan”, American Journal of Epidemiology, Vol. 178, No. 1, June 2013; N. E. Avis, “Duration of Menopausal Vasomotor Symptoms Over the Menopause transition, JAMA Intern Med., Vol. 174, No. 4, 2015; and S. D. Harlow, “Executive Summary of the Stage of Reproductive Aging Workshop +10: Addressing the Unfinished Agenda of Staging Reproductive Aging”, J Clin Endocrinol Metab, Vol. 97, No. 4, April 2012, each of which are fully incorporated herein by reference for their general and specific teachings.

In some embodiments, the processor circuitry 100 can further execute the instructions to identify the menopause-related anomaly in the set of features. As previously described, each of the at least one ML models can include a plurality of different patterns of the set of features which are indicative of different states of menopause. The menopause-related anomaly can include a divergence from at least one of the baseline pattern of the set of features for the user and/or the general population trends. For example, the output of the second ML model, as described above, can be an indicator of the menopause-related anomaly associated with the divergence, such as a reconstruction error of reconstructed input features as compared to the input features. In various embodiments, the second ML model is used to generate pseudo-features from the set of features and to identify the menopause related anomaly from an output of the second ML model and, optionally, at least one of the pseudo-features and the menopause related-anomaly can be input to the first ML model.

Example menopause-related anomalies can include a change in a feature or multiple features that are different from the baseline pattern or general population trends and transitions between menopause states at unexpected times based on the general population trends. For example, the menopause-related anomalies can include patterns that are unexpected based on the general population trends or the baseline pattern, which can indicate early menopause transition for the user. Specific example anomalies can include changes in sleep disruption, menstrual cycle, hormone levels, among other pattern changes, and which can indicate a transition between menopause states at a different (e.g., earlier) time than expected based on the baseline pattern and/or general population trends. In some embodiments, the menopause-related anomalies can include or be associated with multiple features, such as anomalies in patterns between the features and where analysis of each of the multiple features individually may not result in identifying the menopause-related anomaly. As an example, the baseline pattern or general population trends can identify that a normal pattern of heart beats is between 60-70 beats per minute and a normal pattern of skin temperature is between 80 and 70 degrees. With respect to each other, a normal pattern of heart beats compared to skin temperature can include lower skin temperature with lower heart beats. In such an example, a menopause-related anomaly can be identified when the heart beat is raised (within the normal range) while skin temperature is not raised (but may still be within the normal range). In addition and/or alternatively, the anomalies can be associated with one feature, such as a change in a feature that is above a threshold or outside a normal change (e.g., increase of 10 percent in heart rate, increase by 50 percent of hot flash events). The data message communicated to the user can flag the menopause-related anomaly and direct the user to contact a profession, such as a physician.

ML models can include data models which estimate or provide an output based on input data. Various ML frameworks are available from multiple providers which provide open-source ML datasets and tools to enable developers to design, train, validate, and deploy ML models, such as AI/ML processors. AI/ML processors (sometimes referred to as hardware accelerators (MLAs), or Neural Processing Units (NPUs)) can accelerate processing of ML models. ML processors are integrated circuits (ASICs) that can have multi-core designs and employ precision processing with optimized dataflow architectures and memory use to accelerate calculation and increase computational throughput when processing ML models.

Example ML models include artificial neural network, support vector machine (SVM), deep learning, cluster, and/or other models. An artificial neural network can estimate a function(s) that depends on inputs. In some embodiments, one or more layers of artificial neurons can receive input data and generate output data. The input data can comprise the set of features and the output data can include an indicator of current menopause state and/or indicator of a menopause-related anomaly. Neural networks can comprise networks such as, but not limited to, learning networks (e.g., deep, deep structured, hierarchical, and the like), convolutional, auto-type networks (e.g., autoencoder, auto-associator), Diablo networks, and neural network models (e.g., feedforward, recurrent).

An SVM can utilize a linear classification. This classification can act to separate the data points into classes based on distance of the data points from a hyperplane. In some embodiments, the hyperplane is arranged to maximize the distances from the hyperplane to the nearest data points on either side of the hyperplane. This arrangement can group points located on opposite sides of the hyperplane into different classes. However, in some embodiments, the SVM can comprise a nonlinear classification that separates the data points with a hyperplane in a transformed feature space. The transformed feature space can be determined by one or more kernel functions, including nonlinear kernel functions. In some embodiments, the SVM is a multiclass SVM that separates data points into more than two classes, which can reduce a multiclass problem into multiple binary classification problems.

In some embodiments, a deep learning model can include models such as, but not limited to, convolutional networks (e.g., deep belief, neural), belief networks, Boltzmann machines, deep coding networks, stacked autoencoders, stacking networks (e.g., deep or tensor deep), hierarchical-deep models, deep kernel machines, and the like. It will be understood that such embodiments can comprise variants and/or combinations of the above-noted example networks.

In some embodiments, the ML model(s) can comprise a clustering method(s), which can comprise hierarchical clustering, k-means clustering, density-based clustering, and the like. In some embodiments, the hierarchical clustering can be used to construct a hierarchy of clusters of the set of features. In some embodiments, the hierarchical clustering utilizes a “bottom up” approach (e.g., agglomerative) wherein each data point starts in its own cluster, and pairs of clusters are merged at progressively higher levels of the hierarchy. However, in some embodiments, the hierarchical clustering utilizes a top-down approach in which all data points start in one cluster, and then clusters are split at progressively lower levels of the hierarchy.

In some embodiments, the k-means clustering implementation can comprise placing the set of features into k clusters, where k is an integer equal or greater than two. Via such clustering, each data point belongs to a cluster having a mean that is closer to the data point than any means of the other clusters. However, in some embodiments, a ML model can comprise density-based clustering, which can be used to group together data points that are close to one another, while identifying as outliers any data points that are far away from other data points.

In some embodiments, a ML model can comprises a mean-shift analysis that can be used to determine the maxima of a density function based on discrete data sampled from that function.

In some embodiments, a ML model can comprise structured prediction techniques and/or structured learning techniques. Such techniques can be used to predict structured objects and/or structured data, such as structured sets of features and/or sensor data. In some embodiments, such structured prediction and/or structured learning techniques can comprise graphical models, probabilistic graphical models, sequence labeling, conditional random fields, parsing, collective classification, bipartite matching, Bayesian networks or models, and the like. It will be understood that such examples comprise variants and/or combinations of the above-noted example techniques.

In some embodiments, a ML model can comprise anomaly detection and/or outlier detection that can be used to identify data that does not conform to an expected pattern or are otherwise distinct from other data in a dataset.

For example, extracted feature sets can be input into the first ML model and used to identify the pattern within the set of features which is indicative of the probability (or other confidence score) of the user being in a state of menopause. In some embodiments, the user can exhibit patterns for more than one state of menopause, with different probabilities or other confidence scores. In some embodiments, the extracted features are input to the second ML model to generate pseudo-features (and optionally, an identified menopause-related anomaly) which are input to the first ML model. Based on the pattern and the input (e.g., the pseudo-features), the first ML model can output a probability of the state of menopause for the user and/or identify an anomaly in the set of features. Similarly, the set of features can be input to the second ML model, which can identify the menopause-related anomaly. For example, the menopause-related anomaly can be identified based on a reconstruction error of reconstructed input features as compared to the input features.

In some embodiments, each of the first and second ML models can include a plurality of sub-models used to generate sub-patterns for different subsets of features of the set of features, and the instructions are executable or executed to apply the at least one ML model to the longitudinal dataset to obtain a confidence score for each of the plurality of sub-models and predict the current state of menopause or identify the menopause-related anomaly based on the confidence score for each of the plurality of sub-models based on the generated sub-patterns. Each of the confidence scores can be weighted based on at least one of a level of predictiveness of the subset of features and a reliability of sensor signals associated with the subset of features. The confidence scores can include probabilities and/or reconstruction errors (e.g., similarity or divergence of input from reconstructed input), among other scores.

In some embodiments, the current state of menopause that is predicted can be based on past predicted states. The states generally follow the order of pre-menopause, menopause transition (e.g., perimenopause and/or early and late menopause), and post-menopause. If the user was previously predicted to be in post-menopause for the past few months, the at least one ML model can weigh heavily against predicting the user is in the menopause transition (or other menopause states). In some embodiments, the menopause-related anomaly identified can include an unexpected change in the predicted state, such as skipping a state and/or entering a state early and/or the current state otherwise being not compatible with a past predicted state (e.g., go from early post-menopause to menopause transition), and/or an accelerated transition between states. In some embodiments, pseudo-features and/or the menopause-related anomaly, identified using the second ML model, can be input as the set of features to the first ML model.

As noted above, the features can be associated with sensor signals obtained over different time windows (sometimes herein interchangeably referred to as “different sample times” or “time periods”). After extracting the features, the processor circuitry 100 can time-align the features to a common time window. For example, the sensor signals can be obtained by the sensor circuitry at or based on the different time windows. The processor circuitry 100 can align the extracted features of the set of features to a common time window and to predict the current state of menopause for the user based on a plurality of past predicted states of menopause for the user. In some embodiments, the processor circuitry 100 can weigh each of the extracted features based on an impact of the extracted features on the probability of the state of menopause. For example, the different weights can be dependent on an impact of the feature to the current state of menopause for the user. In some embodiments, as described above, the weighted features can be compressed into pseudo-features using the second ML model and input to the first ML model.

The following describes two example approaches for combining the set of features from the sensor circuitry and/or with self-reported measures. The approaches can be referred to as “late fusion” and “early fusion” for ease of reference. In late fusion, the at least one ML model includes sub-models associated with different feature groups of the set of features and each sub-model provides a confidence score. Once each sub-model provides the confidence score, a final weight is computed by combining the confidence scores of the sub-models and producing a single confidence score. One advantage of the late fusion approach is that it has lower complexity when adapting on-the-fly. In some instances, the processor circuitry 100 can adapt the sub-models based on the user inputs in real-time, such feedback data and/or sensor signals. In addition, the final fusion weight can be adjusted and reweighted based on feedback data to bias the prediction towards weighting the reliable sensor signals for the current (e.g., real-time) session.

For example, and in accordance with the late fusion approach, the at least one ML model includes a plurality of sub-models, and each of the plurality of sub-models are associated with a feature or a subset of features of the plurality and are each used to provide an output score indicative of the confidence (e.g., probability) of the current state of menopause for the user based on the extracted features from the respective sensor signal obtained by the sensor circuitry. The processor circuitry 100 can combine the output scores from the plurality of sub-models to identify the probability or confidence score of the current state of menopause for the user and/or to identify an anomaly in the set of features.

In early fusion, the features are combined to form a vector at each time-point. The vector of combined features is used as input to the at least one ML model which produces a probability score or other confidence score. A threshold is used to predict the current state of menopause for the user. In this approach, a decision tree structure can used to combine multiple features. The early fusion approach allows for several decision paths to be used, which adds robustness and redundancy to the decisions. The decisions at each time-point (instantaneous decisions) can be combined by detecting consecutive current state predictions and converting to time-regions. The advantage of the early fusion approach is tighter feature integration and which allows for higher accuracy by better exploiting feature relations. In some embodiments, the decision tree structure can include a binary tree structure. For example, the features can be used by a binary tree structure in successive thresholds (e.g., higher or lower threshold indicate different weights), which are learned from the data. The thresholds can be adjusted manually and/or automatically by the at least one ML model.

For example, and in accordance with the early fusion approach, the processor circuitry 100 can combine the extracted features from the plurality of sensor signals into a vector and input the vector to the at least one ML model to produce an output score indicative of the probability or a confidence score of the user being in a state of menopause. The processor circuitry 100 can compare the output score to a threshold and predicts the current state of menopause if the output score is above the threshold and is not the current state if below the threshold for both the early and late fusions. In some embodiments, the vectors can be generated and/or compressed using the second ML model.

The processor circuitry 100 can track the set of features and the predicted current states of menopause over time. For example, the processor circuitry 100 can generate and/or use a decision tree structure to combine the extracted features and to produce an output score based on the combined extracted features. In some embodiments, the processor circuitry 100 can predict the current state of menopause at a plurality of time points based on output scores, detect consecutive predicted states, and convert the consecutive predicted states into a current state region. A decision tree, as used herein, includes or refers to a data structure that forms part of, or includes the ML model that represents different decisions as branches to reach outputs or decisions that are represented as leaves. The decision tree can be used to predict the probability (or other confidence score) of a state of menopause based on a plurality of input features extracted from sensor data and/or self-reported measures, and can be used to provide fusion of a set of complex rules as multiple paths.

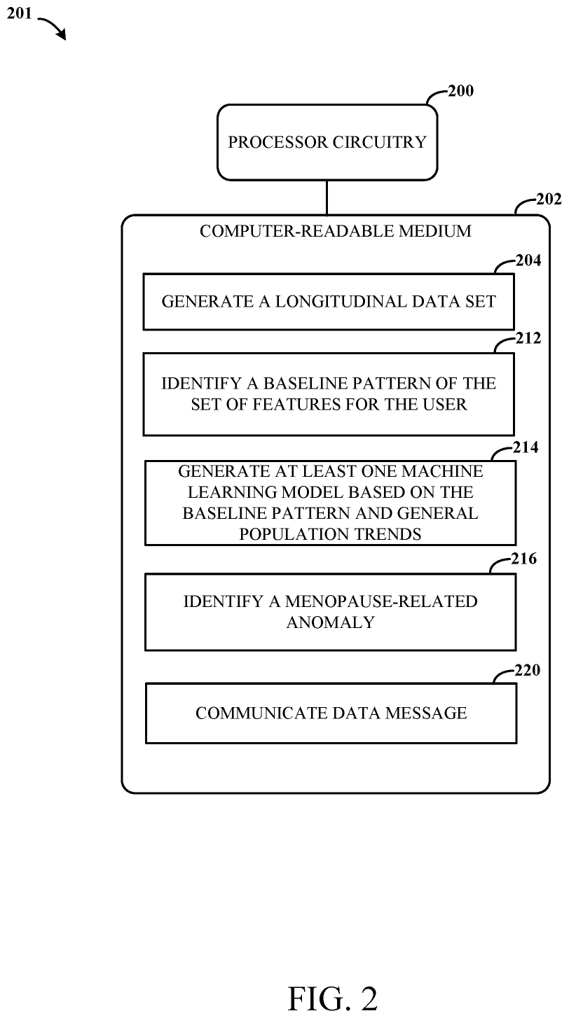

FIG. 2 illustrates an example computing device including non-transitory computer-readable medium storing executable code, in accordance with the present disclosure. Similar to FIG. 1, the processor circuitry 200 can form part of a computing device 201. The computing device 201 includes the processor circuitry 200 and computer-readable medium 202 storing a set of instructions 204, 212, 214, 216, 218, 220. In some embodiments, the computing device 101 of FIG. 1 can further include the instructions 204, 212, 214, 216, 218, 220 illustrated by FIG. 2 and/or vice versa.

At 204, the processor circuitry 200 can execute instructions to generate a longitudinal dataset of a set of features from tracked physical measurements of a user as received from sensor circuitry. At 212, the processor circuitry 200 can identify a baseline pattern of the set of features for the user using at least a portion of the longitudinal dataset of the set of feature, and at 214, can generate at least one ML model based on the baseline pattern of the set of features and general population trends of a demographic population associated with the user. A baseline pattern, as used herein, can include and/or refer to values or ranges of values and patterns which are observed in the user, and which may be different from general population trends in some instances. In some embodiments, the at least one ML model includes a first ML model used to predict a current state of menopause for the user and a second ML model used to generate pseudo-features from the set of features and to identify menopause-related anomalies. At 216, the processor circuitry 200 can identify a menopause-related anomaly in the set of features using the at least one ML model (e.g., the second ML model) based on a change (e.g., deviation) from the baseline pattern of the set of features and the general population trends. In some embodiments, the menopause-related anomaly can be identified based on a reconstruction error of reconstructed inputs as compared to input features determined using an autoencoder, as further described herein.

In some embodiments, the processor circuitry 200 can use the set of features that are associated with the menopause-related anomaly to identify a pattern within the set of features indicative of the user being in a state of menopause. For example, the menopause-related anomaly can be used as an input to the first ML model, along with the set of features, and used to predict the current state of menopause. As further described below, the input set of features to the first ML model can include the pseudo-features from a bottleneck layer of the second ML model (e.g., compressed versions of the set of features) and/or the output reconstruction error. In other embodiments and/or in addition, the output of the second ML model can be separately analyzed to identify the pattern by identifying a subset of the set of features associated with the anomaly and identifying the pattern from the subset of the set of features.

At 220, the processor circuitry 200 can communicate a data message indicative of the menopause-related anomaly to the user. The data message can be communicated by the computing device 201 (or the computing device 101) providing the data message on the display of the computing device 201 (and/or computing device 101 of FIG. 1) and/or communicating the data message to another device for display. For example, the processor circuitry 200 can automatically send the data message to the user indicative of an issue associated with the menopause-related anomaly and notify the user to contact a professional (e.g., a physician). As previously described, the menopause-related anomaly can include a normal pattern of features is happening early and/or an abnormal pattern of features is occurring (e.g., changes in sleep disruption (or other feature) outside normal or baseline patterns, having events that are not otherwise expected (e.g., would expect sleep pattern to change if transitioning but all other features indicate transition between states), and/or patterns between different features that are not expected or are outside the normal/baseline). In some embodiments, the menopause-related anomaly includes an abnormal transition between menopause states.

Similar to the computing device 101 of FIG. 1, the processor circuitry 200 can further execute instructions to predict a current state of menopause for the user based on the identified pattern, wherein the communicated data message is indicative of the menopause-related anomaly and the current state of menopause. As previously described, the at least one ML model can include a first ML model indicative of a predicted current menopause state (e.g., a prediction of a menopause state for the user) and a second ML model indicative of expected patterns of features and used to generate the pseudo-features, with deviations from the patterns used to identify menopause-related anomalies. The processor circuitry 100 can predict the current state of menopause using the first ML model that identifies the pattern within the set of features indicative of a probability of the user being in the state of menopause, such as by using the pseudo-features and, optionally, the menopause-related anomaly to identify the pattern.

In various embodiments, the processor circuitry 200 can actively trigger self-reported measures from the user. For example, the processor circuitry 200 can provide a data message to the user to get feedback data and/or to otherwise confirm the predicted current state of menopause and/or get additional information on the identified menopause-related anomaly. In some instances, health or other issues other than menopause can cause changes in features tracked by the sensor circuitry. For example, changes in the features can be caused by cancer or other diseases or disorders, pregnancy, medicine (e.g., birth control pills or other medicine), and other lifestyle changes (e.g., dietary changes, exercise changes, weight changes, changes in stress or emotional state). The feedback data can be used to confirm the correct state of menopause is predicted and/or for other purposes, such as revising the at least one ML model. As an example, the weights provided to different features can be adjusted based on the feedback data. Using a specific example, a user may have a disorder that causes irregular menstrual cycles and/or otherwise have a baseline pattern that includes irregular menstrual cycles. The first and/or second ML models can be updated to provide a low (or no) weight to the menstrual cycle for the user in response to the feedback data indicative of irregular menstrual cycles not being tied to and/or predictive of (or is less predictive than an average user) to the menopause state.

In various embodiments, the processor circuitry 200 (and/or the processor circuitry 100) can be used to observe passive perturbations and/or cause active perturbations. For example, the processor circuitry 200 can identify perturbations are occurring and observe changes in patterns of measured features for the user in response. As some examples, passive perturbations can include or be associated with natural cycles, such as menstrual cycles and phases thereof, time of the day (e.g., night verses awake), sleep states and/or transitions (e.g., REM sleep), among other natural cycles. As an example, the pattern of features observed can be different for the different natural cycles. In some embodiments, the processor circuitry 200 can actively cause the perturbation and observe the measured features in response. Example active perturbations include an audio output, a visual or light output, a data message provided to the user, among others. For example, the processor circuitry 200 can cause a sensory output (e.g., audio tone, visual indicator, among others) to the user and observe the changes in features measured from the user in response. In some embodiments, the processor circuitry 200 forms part of the computing device 201 which can include speakers and can output an audio tone and/or include a screen or other light source which can output a visual indicator. In other embodiments, the processor circuitry 200 can include the screen and provide the data message to the user on the screen directing the user to perform a task, and observes the changes in features measured from the user in response. In other embodiments, the processor circuitry 200 can output a data message to another computing device to perform the active perturbation, such as to a wearable device and/or cooling device (e.g., provide cooling to the user).

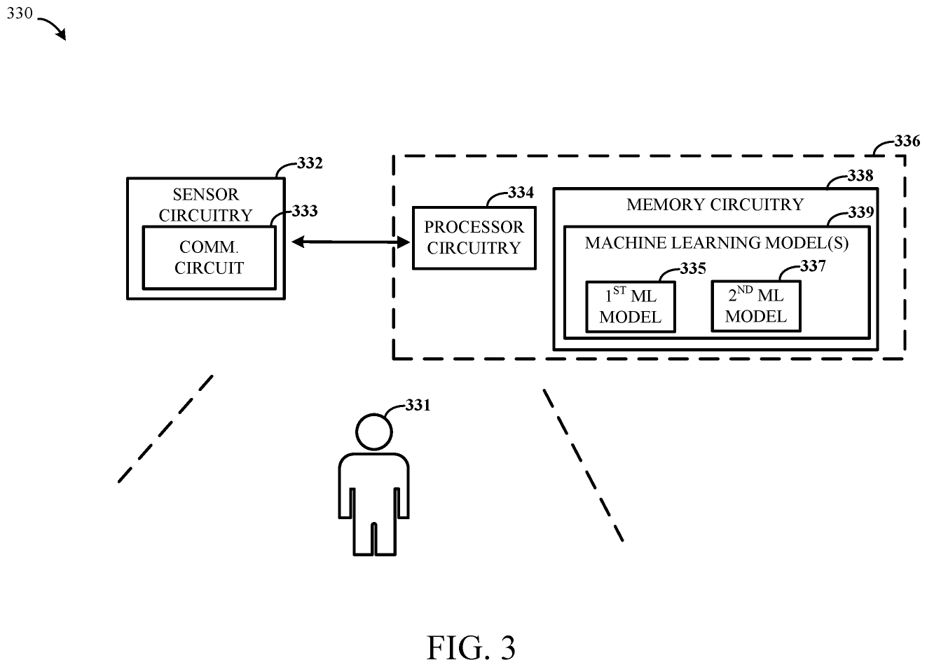

FIG. 3 illustrates an example system including sensor circuitry and processor circuitry, in accordance with the present disclosure.

The sensor circuitry 332 is configured to obtain sensor signals indicative of physical measures associated with a user 331. The sensor circuitry 332 can include communication circuitry 333 to communicate the physical measures. In some embodiments, the sensor circuitry 332 includes a wearable device and/or sensor, such as a smart watch or smart ring worn by the user 331. In other embodiments, the sensor circuitry 332 includes a plurality of sensors, such as a plurality of wearable devices or other sensors that may or may not be worn by the user 331.

In some embodiments, the processor circuitry 334 forms part of a computing device 336. The computing device 336 includes the memory circuitry 338 that stores the at least one ML model 339 and the processor circuitry 334. In some embodiments, the computing device 336 can include the computing device 101 of FIG. 1 and/or computing device 201 of FIG. 2. However embodiments are not so limited and the processor circuitry 334 and memory circuitry 338 can be distributed from one another. For example, although FIG. 3 illustrates a single processor circuitry 334 and memory circuitry 338, embodiments are not so limited and can be directed to devices and/or systems with multiple processor circuits and multiple memory circuits. The instructions can be distributed and stored across the multiple memory circuits and can be distributed and executed by the multiple processor circuits.

The processor circuitry 334 is configured to generate a longitudinal dataset of a set of features from the physical measurements of the user as tracked over time, identify a baseline pattern of the set of features for the user using at least a portion of the longitudinal dataset of the set of features, and generate at least one ML model 339 based on the baseline pattern of the set of features and general population trends of a demographic population associated with the user. In some embodiments, generating the at least ML model 339 can include accessing the at least one ML 339 from the memory circuitry 338. In some embodiments, generating the at least one ML model 339 can include constructing and training the at least one ML model 339 and storing the trained ML model on the memory circuitry 339. As previously described, the at least one ML model 339 can include a first ML model 335 and a second ML model 337, which can be demographic ML models and/or user-specific ML models.

In some embodiments, the system 330 further includes input circuitry to receive information from the user 331. For example, the computing device 336 can include the input circuitry used to receive the information and the set of features can include the received information, such as the previously described self-reported measures. The self-reported measures can be used to supplement the sensor data from the sensor circuitry 332 and/or to provide confirmation or feedback data for predicting the current state of menopause and/or the identified menopause-related anomaly.

The processor circuitry 334 is further configured to identify a menopause-related anomaly in the set of features using the at least one ML model 339 and based on a change from the baseline pattern of the set of features and the general population trends, identify a pattern within the set of features indicative of a probability of the user being in a state of menopause using the at least one ML model 339 and at least one of the set of features and the menopause-related anomaly, and communicate a data message indicative of the menopause-related anomaly to the user.

As previously described, the pattern within the set of features can be identified by analyzing the set of features based on the output from the second ML model 337 and/or with the menopause-related anomaly provided as an input to the first ML model 335. In some embodiments, pseudo-features generated using the second ML model 337 and the output of the second ML model 337 (e.g., the reconstruction error) are used as the input set of features to the first ML model 335. In some embodiments, the first ML model 335 is indicative of the probability of the user being in the state of menopause and the second ML model 337 includes an encoder/decoder pair applied to generate the pseudo-features from the set of features and to identify the menopause-related anomaly from a reconstruction error determined using the pseudo-features and the set of features, as further described below. The processor circuitry 334 can apply the first ML model 335 to predict a current state of menopause for the user based on an identified pattern within the set of features (e.g., the pseudo features) indicative of a probability of the user being in the state of menopause. In some embodiments, the data message communicated by the processor circuitry 334 can be indicative of at least one of the menopause-related anomaly and the predicted current state of menopause for the user. In some embodiments, the processor circuitry 334 can communicate another data message indicative of the current state of menopause or communicate the data message which is indicative of both the predicted current state of menopause and the menopause-related anomaly. The current state, as previously described, can be associated with a transition to or from a state selected from pre-menopause, menopause transition, and post-menopause. The processor circuitry 334 can predict the current state of menopause for the user based on the identified pattern, a plurality of past predicted states of menopause for the user, the pseudo-features, and/or the menopause-related anomaly, as previously described.

In some embodiments, the processor circuitry 334 is configured to identify the menopause-related anomaly in the set of features using the at least one ML model 339 and based on a deviation from the baseline pattern and general population trends of the demographic population associated with the user. For example, the processor circuitry 334 can identify the menopause-related anomaly in the set of features using the at least one ML model 339 that includes a neural network data model with hidden states to identify the baseline pattern and the anomaly is identified from the baseline pattern using the longitudinal dataset, wherein the baseline pattern includes a plurality of sub-patterns associated with the set of features (e.g., different sub-pattern for different feature subsets and which can be for a single feature or for groups of features). In some embodiments, the at least one ML model 339 includes an autoencoder having an encoder/decoder pair and which generates pseudo-features from the set of features (e.g., each pseudo-feature is a compressed from of a subset of features), as further described below.

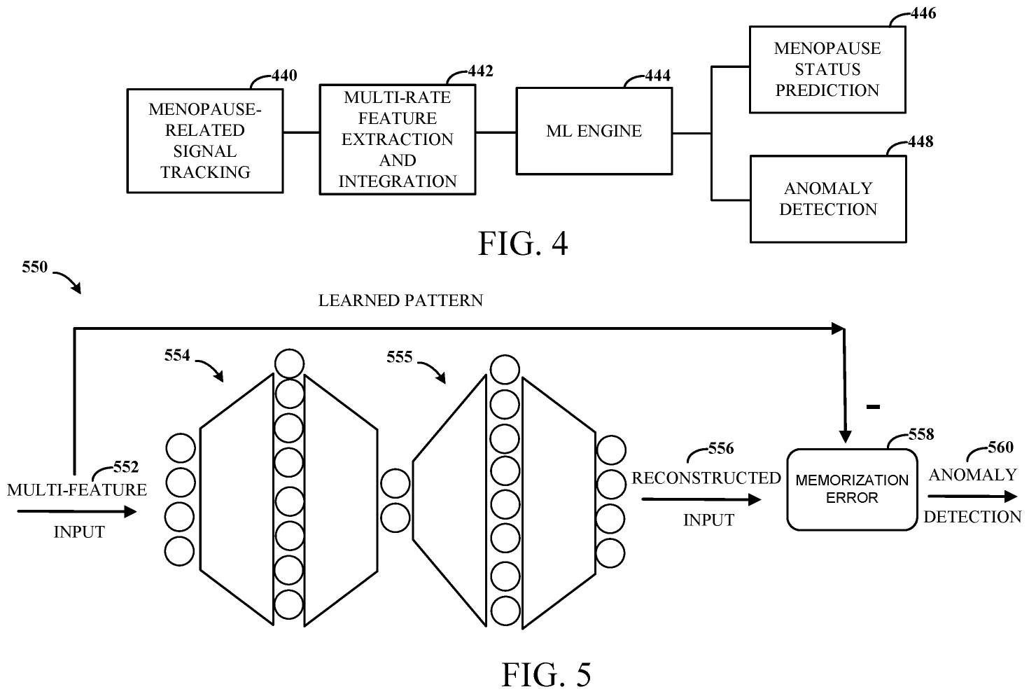

FIG. 4 illustrates an example multi-modal bio-behavioral tracking system, in accordance with the present disclosure. As shown, the system can track the menopause-related signals, at 440. Sensor signals can be obtained to track a diverse feature set having collection diversity, and can be supplemented with self-reported measures, sometimes referred to as the multi-rate features.

The system can extract the multi-rate features and integrate the multi-rate features, at 442. As previously described, the set of features can have collection diversity and feature diversity. The following describes example processes for extracting and integrating the features.

For collection diversity, the features can be grouped into subsets. Each subset represents information collected at asynchronous time windows. For example, the one subset contains features from sensor signals on second resolution such as temperature, PPG and skin conductance; another subset contains features from sensor signals and clinical data on weekly resolution such as skin pH levels and so on. These groups are collected asynchronously with respect to each other.

The feature grouping approach has several advantages for both learning and prediction including: (1) train on different data-sources independently; (2) minimize the data required to train each model; (3) use separate machine learning for each feature group; (4) handle missing features (e.g., not collected); and (5) collect data asynchronously.

For feature extraction, multi-sensor low-level objective features can be leveraged. Features include heart rate, night temperature, etc., and subjective features. Features include sleep and hot-flash indicators. In some embodiments, at least some of the feature extraction can be implemented in accordance with US Publication No. 20200013511A1, entitled “Systems and Methods Involving Predictive Modeling of Hot Flashes”, published on Jan. 9, 2020, which is fully incorporated herein by reference in its entirety for its general and specific teachings.

The extracted and integrated features are input to a ML engine 444, which can output a predicted menopause state 446 and/or an identified menopause-related anomaly 448 and/or indicators thereof. Accurate menopause modeling can involve bio-behavioral features. The ML engine 444 can include processor circuitry that executes at least one ML model, such as the first ML model and the second ML model as previously described. The ML engine 444 can construct, train, store, and/or revise the at least one ML models. Example bio-behavioral features include sleep disturbances, vaginal dryness measures, pH levels and other relevant features. The above described menopause state prediction, at 446, can rely on learning the relevant bio-signatures from data (e.g., feature sets). Menopause state prediction has specific properties to be considered in modeling including features that impact the menopause state and the menopause event rarity.

Menopause state is affected by (a) what is normal or baseline for each user and (b) divergence from the population. Longitudinally tracked features can be used to capture what is normal or baseline for each user. Changes from baseline can be measured to model menopause state changes. Both the relative and absolute feature values can be used to model user changes from the baseline and divergence from the population mean. These are used as features for menopause state prediction.

A challenge for menopause modeling and prediction is that it is a rare event and happens once for each user. To model the different women's reproductive stages (e.g., pre-menopause, menopause transition [early, late], post-menopause [early, late]), cross-sectional learning can be applied here. The system learns which features are more informative and apply the information to a new user.

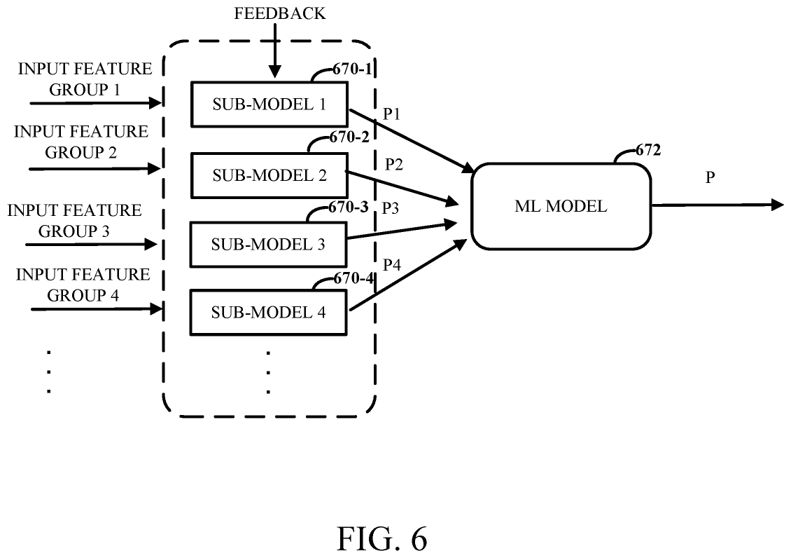

In various embodiments, as further shown by FIG. 6, features are grouped and different sub-models are learned for each subset of features. The different sub-models provide a confidence score for pre-menopause, menopause transition [early, late], and post-menopause [early, late]. Each group contributes to the final score. Each confidence output is weighted by the predictive power and reliability of the feature subset. Grouping provides redundancy and robustness to missing or noisy features, and/or can enable training from independent data collections.

Various embodiments include sequential processing of the features and use of past predicted states. Menopause states for women can happen in a specific sequence including pre-menopause, menopause transition [early, late], and post-menopause [early, late] and may always appear in this order. Past state predictions are exploited to limit the possibilities of future state predictions.

As noted above, various embodiments are directed to identifying menopause-related anomalies in the normally occurring menopause transition, at 448. Identifying menopause-related anomalies in the menopause transition uses knowledge and information of what is the baseline for each user. The ML engine 444 can learn what is the normal or baseline pattern for menopause states for each user based on past physical measures and detects any divergence from the baseline. For example, a particular user (e.g., women) can have different patterns for particular features of the set of features as a baseline during the pre-menopause state, which may differ from general population trends. As a specific example, a particular user can have irregular menstrual periods and a number of sleep disturbances when in the pre-menopause state, which can be irregular from general population trends.

The divergence approach can work well when one or few features are used. On the other hand, learning baselines for each user based on past data may not work as well with high-dimensional data, thus, not suitable for multi-sensor complex feature relations. In some embodiments, the ML engine 444 (e.g., a processing circuitry of a computing device) can implement an autoencoder which models high-dimensional, generic, and diverse features. The approach learns how to use all the features available jointly, represent the information with a low dimensional hidden state (referred to as an autoencoder bottleneck) and finally using the hidden information to reconstruct the original features as shown in FIG. 5.

FIG. 5 illustrates an example autoencoder for memorizing patterns of input features using longitudinal data, in accordance with the present disclosure. The autoencoder 550 can include computer-readable instructions which are stored on memory circuitry and/or executed by processor circuitry, such as the processor circuitries 100, 200, 334 illustrated by any of FIGS. 1-4. The autoencoder 550 can learn from a few historical data points what are the normal signals using a large set of features (e.g., hundred(s), thousand(s)) as input, e.g., the multi-feature input 552. The multi-feature input 552 can include the previously described set of features, such as the multi-rate features. The autoencoder 550 is used to learn and memorizes the typical patterns, and includes an encoder/decoder pair. The encoder 554 of the encoder/decoder pair can compress the input features of the set of features to a low dimensional hidden state which includes a reduced number of features as compared to the input features 552. For example, the features available are jointly represented as information with a low dimensional hidden state that includes subsets of the set of features which are combined into pseudo-features of a reduced number from the input feature set, which can be referred to as an autoencoder bottleneck. The input features are compressed into the reduced number of pseudo-features by applying a combinatorial function to the subsets of the set of features, with the combinatorial functions being learned during the training of the autoencoder 550 by comparing the input features to the reconstructed features and adjusting the combinatorial functions to reduce a reconstruction error. As such, the bottleneck layer can include fewer pseudo-features than the input features, which are output to the decoder 555. In some embodiments, the pseudo-features can be used as the input set of features to the first ML model. The encoder 554 thereby reduces the size of the input set of features 552 and reduces the size of the input task by combining subsets of the input set of features into pseudo-features that represent the respective subsets of the input set of features.

The decoder 555 uses the hidden information to reconstruct the original input features, as shown at 556. The hidden information can include the pseudo-features and the combinatorial functions used to compress or generate the pseudo-features. With typical multidimensional data, the autoencoder 550 can reconstruct the input features with low reconstruction error, at shown at 558. The reconstruction error can include an error of the reconstructed input features 556 (as reconstructed using the pseudo-features and the combinatorial functions) as compared to the input features 552. If a new unseen signal appears (e.g., an unexpected feature value or pattern), the autoencoder 550 reconstructs the features with a large error, which can be used to identify and/or detect a menopause-related anomaly, at 560. The autoencoder 550 thereby compresses high dimensional features into low dimensional features, and learn latent anomalous information of multiple signals.

As described above, in some embodiments, the pseudo-features can be used on the input set of the features to the first ML model indicative of a probability of the user being in a state of menopause, which can be referred to as a predictive ML model. For example, the pseudo-features can be represented as a vector (e.g., a vector of the output pseudo-features) that is input to the first ML model. By using pseudo-features, which retain information from the subset of features forming the respective pseudo-feature, the number of inputs to the first ML model is reduced, e.g., reduced dimensionality of data used for predicting the menopause state. The reduced dimensionality of data can improve accuracy of the first ML model and result in increased processing speed and/or reduced processing resources to provide the output of the first ML model (e.g., to predict the current menopause state for the user).

As described above, in some embodiments, the pseudo-features can be represented as a vector and input to the first ML model and/or the input features may be represented as vectors before being input to or by the second ML model. In some embodiments, different vectors can be formed which are representative of different modalities of sensor signals and/or other input data, such as self-reported measures. For example, values of the different sensor signals, which are indicative of different features, can be represented by different vectors with the values being embedded as arrays of numbers. In various embodiments, the vectors may be implemented as described by U.S. Publication 2019/0325342, published on Oct. 24, 2019, and entitled “Embedding Multimodal Content in a Common Non-Euclidean Geometric Space”, which is fully incorporated herein by reference in its entireties for its teachings.

In some embodiments, the autoencoder 550 can include a variational autoencoder (VAE) that identifies a similarity of a plurality of tasks (e.g., the pseudo-features) to an input task (e.g., analyzing the set of features). For example, a VAE can comprise at least one encoder, such as set of encoders which reduce a size of the input tasks to a smaller latent space z, and at least one decoder, such as a set of decoders which reconstructs the latent space z (e.g., a cluster of related tasks which are reduced to the latent space) into an output task representative of the cluster of related tasks. For example, the encoder(s) can encode the set of features into pseudo-features, which are input to the first ML model and used to predict a current menopause state for the user, and the decoder(s) decode the pseudo-features into reconstructed features and to a reconstruction error indicative of a similarity (or error) the reconstructed features as compared to the set of features.

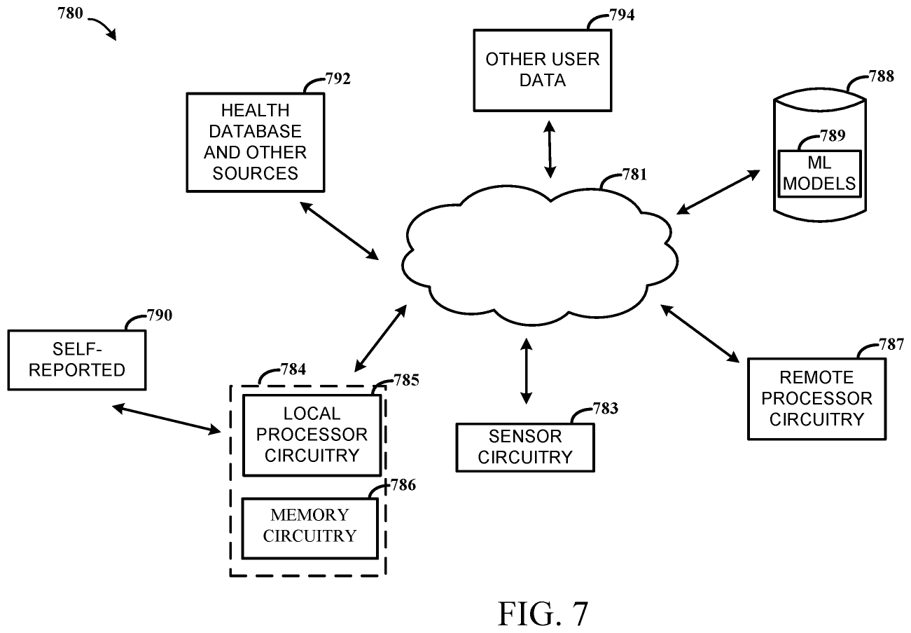

In some embodiments, the menopause-related anomaly can indicate the user is in an unstable menopause state, such as indicating a transition in menopause states or other abnormalities. For example, in a stable state, the encoder 554/decoder 555 can encode a maximum amount of information in the pseudo-features (e.g., retain information from the features) while minimizing the reconstruction error when decoding. In some embodiments, the encoder 554/decoder 555 can be communicated, such as a remotely-located processor circuitry generating the encoder 554/decoder 555 and communicating to a plurality of local processor circuitry, such as illustrated further herein by FIG. 7.

In some embodiments, the autoencoder 550 can depend on at least the features to be processed and in some embodiments can include at least one of a vanilla autoencoder. a VAE, and a VAE for time series anomaly detection. In some embodiments, the encoder 554 can implement a neural network, such as convolution neural network a long short-term memory (LSTM), to convert the received input set of features into the compressed form, e.g., the pseudo features, of the set of features. in the latent space between the encoder 554 and the decoder 555, the pseudo-features can be further processed into a latent code, a most efficient representation of the data representation of the features of a stably state of menopause. in embodiments in which the input to the autoencoder 550 includes a set of features from a user known to be in a stable menopause state, the latent code is defined as the training model. The latent code can then be process (e.g., processed using a neural network) by the decoder 555 to determine reconstructed features which are a representation of the input data. The determined training model can be communicated, for example in some embodiments, such as to other circuitry. In some embodiments, the determined training model can be communicated, to a plurality of processor circuitry associated with different users and used to identify menopause-related anomalies and/or to retrain the training model using data specific to the user.

FIG. 6 illustrates an example of a ML model, in accordance with various embodiments. As described above, a processor circuitry and/or ML engine can use and/or to generate at least one ML model 672 that identifies menopause-related anomalies and/or indicates a probability of a state of menopause for a user. The illustrated ML model 672 can include an implementation of the first ML model and/or second ML model as previously described.

The ML model 672 accepts several different input data, such as 1) sensor signals collected with wearable sensors and/or devices, 2) user provided or self-reported measures, and optionally other data such as 3) environmental sensor signals including temperature, humidity, and so forth, 4) demographic information, and 5) general population trends. The sensor signals can include a plurality of different data, such as HR, motion data, SC and/or temperature, among other data. The ML model 672 can also be informed by scientific discoveries in the literature, such as health web-sites, online feeds, and/or journal articles, that can serve as an additional input. The output of the ML model 672 is the indicator of the predicted current state of menopause and/or identified or detected anomalies.

The inputs are processed according to obtaining a representation that allows the processing circuitry and/or ML engine to learn the patterns of the feature sets with respect to menopause-related anomalies and/or transitioning to or not a different menopause state for the user, and which can include use of different ML processes to generate sub-models 670-1, 670-2, 670-3, 670-4. The sub-models 670-1, 670-2, 670-3, 670-4 may be generated using a single ML system and/or ML engine and/or across multiple ML systems and/or ML engines (e.g., different ML engines generate a respective sub-model). For example, and as previously described, the features can be grouped into different feature subsets. In some embodiments, each feature subset represents information collected at asynchronous time-periods. For example, the one subset contains features from sensor signals on second resolution such as temperature, PPG and skin conductance; another subset contains features from sensor signals and clinical data on weekly resolution such as skin pH levels and so on. These subsets are collected asynchronously with respect to each other. In other embodiments, each feature subset can be associated with a different pattern, as illustrated by the sub-models 670-1, 670-2, 670-3, 670-4 which generate the different patterns. Different ML processes can be incorporated in the ML model 672 depending on the input categories of data. The ML model 672 can be used to build sub-models 670-1, 670-2, 670-3, 670-4 between the inputs and the outputs which are current and/or future probabilities of the current menopause state and/or transitions. Based on the “gold standard” measure of menopause (e.g., clinical classification of menopause, FSH levels, menstrual cycles) and self-reported measures, the features of the ML model 672 are optimized by minimizing a cost function of each of the sub-models 670-1, 670-2, 670-3, 670-4. The cost function is a function that maps the model predicted probability or other confidence score into a real number intuitively representing some “cost” associated with the predicted probability value or other confidence score.

The extracted features can be temporal and/or spectral features representing the physical signals and their specific time pattern, variabilities and frequency content. Temporal features can include statistical measures such as mean, variance, and higher order statistics of the input data in a time frame. Spectral features can be extracted using Fourier transform. Spectral features in one example can be spectral moments, spectral power fractions, spectral power peaks, and spectral power ratios. The features can also be extracted after applying an appropriate transform that facilitates the understanding of the input pattern such as wavelet transform. Features could also include parameters of a model best representing the data in a specific time window. Since the dimension of the input patterns can be very high, statistical methods such as principal component analysis or linear component analysis can be used to transform the features into a lower dimension subspace where a more precise and efficient representation of the input patterns is achieved.

ML methods such as multiple regression, genetic programming, support vector regression, and difference structures of neural networks can be used for this purpose. As an example, a ML neural network with m outputs can be trained for this purpose. However, the output layer can consist of m nodes with logistic activation functions so that each output would be between 0 and 1 as an example. The cost function to be minimized can be the mean squared error and the optimization algorithm can be set to backpropagation.

In some embodiments, different features can be extracted using NLP techniques and are classified or clustered into several groups according to their similarity. In an example, different events can be classified into predefined classes using algorithms such as centroid categorization, naive Bayes, etc.

In another example, the features can be clustered using partitioning algorithms such as K-Means or hierarchical algorithms including agglomerative and divisive approaches. The ML model 672 gives the probability or other confidence score of the identified anomaly and/or current menopause state for the user based on the observed and/or collected data. A simple example is the logistic regression model which defines a linear decision boundary between the training samples associated with a state transition and those that are not. A more complex model can be built when there is a more complex or non-linear relationship between the inputs and output. A deep neural network such as a MLP can be used for this purpose.

To calculate the optimum network parameters (weights and biases in the case of neural networks), an optimization operation, such as backpropagation, can be used which can be done in batch or incremental style. In the batch mode, all available training data are provided to the network to calculate the optimum parameters, while in the incremental style, the parameters are updated each time a training sample is presented to the network.

The optimization of the model parameters can be done by minimizing a cost function. An example of the cost function is the cross-entropy error which the system defines between the estimated probabilities and the “true” state distribution. Given a dataset of N training samples the cross-entropy cost function is defined as follows:

$$J = - {\sum }_{i = 1}^{N} {t}_{i} log ( {y}_{i} ) + ( 1 - {t}_{i} ) log ( 1 - {y}_{i} )$$

where tᵢ is the current menopause state probability for training sample i that could be either 0 or 1, yᵢ is the predicted probability which can take any value between 0 and 1. Minimizing the negative log likelihood cost function is equivalent to maximizing the likelihood of the correct probability. As may be appreciated, other values may be used and embodiments are not limited to using values between 0 to 1.

During training, the cost function is minimized by tuning the model parameters so that the inputs corresponding to the current menopause state and/or anomaly results in an output probability of close to 1 and inputs that are not associated with the current menopause state and/or anomaly results in an output probability of close to 0.

The built model, when fed by new inputs, outputs the probability of the current menopause state and/or anomaly which could vary from 0 to 1. The model is updated over time based on the new user inputs and/or feedback data, and sensor data associated with menopause.

Other ML models that can be used for this purpose can include naive Bayes, probabilistic decision tree and probabilistic support vector machines classifiers. Other structures of neural networks can also be incorporated such as recurrent neural networks, radial basis neural networks, etc.

FIG. 7 illustrates an example system for predicting menopause states and identifying menopause-related anomalies, in accordance with various embodiments.

As shown, a variety of computing resources 783, 784, 787, 788, 790, 792, 794 can form the system 780. In some embodiments, the system 780 can include a cloud computing system or other type of network system which includes a plurality of distributed computing resources. The distributed computing resources can be at different locations, such as local computing resources 784, 783 which are located proximal to the user and remotely-located computing resources 787, 788 which are located remotely from the user. The various computing resources 783, 784, 787, 788, 790, 792, 794 can communicate with one another using data communications over a network 781. For example, the system 780 can be cloud-based and/or can be implemented through other computer systems in alternative architectures, such as a peer-to-peer network.

The system 780 includes a local processor circuitry 785 and local memory circuitry 786, such as those described by FIGS. 1-3, and which can form part of a computing device 784. The computing device 784 can be local to the user, such as a smartphone, laptop, desk computer, or other device that is accessible to the user. The computing device 784 can be in communication with sensor circuitry 783 that is local to the user, such as wearable devices, wearable sensors, and other sensors associated with the environment local to the user. The memory circuitry 786 can store instructions executable by the processor circuitry 785. In some embodiments, the memory circuitry 786 can store a local version of the at least one ML model 789 which can be obtained from a back-end database 788.

In some embodiments, the at least one ML model 789 is stored on the back-end database and processed by remotely by a remotely-located processor circuitry 787. Although one database 788 is illustrated, the system 780 can include a plurality of databases stored on memory circuits which are accessible by a plurality of distributed processor circuits which can train the ML models 789. For example, the remotely-located processor circuitry 787 can construct and train the at least one ML model 789 and provide the trained ML model 789 to the local processor circuitry 785. In such embodiments, either the local processor circuitry 785 or the remotely-located processor circuitry 787 can revise (e.g., retrain) the at least one ML model 789 using tracked sensor data from the sensor circuitry 783, as previously described. In other embodiments, the remotely-located processor circuitry 787 can remotely apply the trained ML model 789 to the longitudinal dataset of the set of features, which are communicated to the remotely-located processor circuitry 787 directly from the sensor circuitry 783 or from the local processor circuitry 785. The at least one ML model 789 can include the previously described models, such as the first ML model and the second ML model.

The system 780 can include other inputs which can be used to generate the ML models 789 and/or revise the trained ML models to be user-specific. In some embodiments, the additional inputs include the self-reported measure 790, as previously described. The self-reported measure 790 can be communicated via input circuitry to the local computing device 784. In some embodiments, the system 780 can include health databases or other sources of health information 792, such as a health application or patient portal to a professional and which allows for feedback information or self-reported measures to be input as a features to the ML models 789. In some embodiments, the other inputs can include features sets from other users of the network 794, such as users who are tracking menopause anomalies and/or states using another local computing device and sensor circuitry associated with the users of the system 780.

Various embodiments are implemented in accordance with the underlying Provisional Application Ser. No. 63/108,987, entitled “Integration of Multimodal Continuous Wearable Sensing Data to Menopause Status and Predict Anomalous Trajectories in Women Approaching Menopause,” filed Nov. 3, 2020, to which benefit is claimed and which is fully incorporated herein by reference in its entirety for its general and specific teachings. For instance, embodiments herein and/or in the Provisional Application can be combined in varying degrees (including wholly). Reference can also be made to the experimental teachings and underlying references provided in the underlying Provisional Application. Embodiments discussed in the Provisional Application are not intended, in any way, to be limiting to the overall technical disclosure, or to any part of the claimed disclosure unless specifically noted.

Although specific examples have been illustrated and described herein, a variety of alternate and/or equivalent implementations can be substituted for the specific examples shown and described without departing from the scope of the present disclosure. This application is intended to cover any adaptations or variations of the specific examples discussed herein.

1. A non-transitory computer-readable storage medium comprising instructions that when executed cause processor circuitry to:
generate a longitudinal dataset of a set of features from tracked physical measurements of a user received from sensor circuitry;
apply at least one machine learning model to the longitudinal dataset of the set of features to identify a pattern within the set of features indicative of a probability of the user being in a state of menopause;
predict a current state of menopause for the user based on the identified pattern; and
communicate a data message indicative of the current state of menopause for the user.

2. The non-transitory computer-readable storage medium of claim 1, further including instructions executable to communicate the data message indicative of the current state of menopause in response to the current state being different than a past predicted state of menopause for the user.

3. The non-transitory computer-readable storage medium of claim 1, wherein the at least one machine learning model includes at least a first machine learning model applied to identify the pattern within the set of features indicative of the probability of the user being in the state of menopause and a second machine learning model, and further including instructions executable to apply the second machine learning model to the set of features to identify a menopause-related anomaly for the user and in the set of features, wherein each of the first machine learning model and the second machine learning model include a plurality of different patterns of the set of features which are indicative of different states of menopause, including the state and the pattern.

4. The non-transitory computer-readable storage medium of claim 3, wherein the second machine learning model is used to generate pseudo-features from the set of features and to identify the menopause related anomaly from an output of the second machine learning model, wherein the output of the second machine learning model includes an indicator of the menopause-related anomaly associated with a divergence from at least one of:
a baseline pattern of the set of features for the user and general population trends.

5. The non-transitory computer-readable storage medium of claim 1, wherein the physical measurements include at least two or more sensor signals indicative of:
heart rate, skin temperature, skin conductance, motion, pH levels, moisture, environmental temperature, environmental humidity, photoplethysmogram (PPG), and combinations thereof.

6. The non-transitory computer-readable storage medium of claim 1, wherein the set of features include features selected from:
menstrual cycle, changes in vaginal characteristics, changes in skin characteristic, hot flash events, sleep disturbances, autonomic nervous system function, heart rate variability, temperature, and combinations thereof.

7. The non-transitory computer-readable storage medium of claim 1, further including instructions executable to:
train the at least one machine learning model using input data including general population trends of features and demographic information associated with the user and expected outputs of the at least one machine learning model including at least one indicator of a menopause state and a menopause-related anomaly;
revise the at least one machine learning model for the user based on a baseline pattern of the set of features for the user identified using at least a portion of the longitudinal dataset of the set of features; and
identify the pattern within the longitudinal dataset of the set of features indicative of the probability of the user being in the state of menopause based on a change from the baseline pattern of the set of features and the general population trends.

8. The non-transitory computer-readable storage medium of claim 1, wherein the at least one machine learning model and the identified pattern include a plurality of sub-models using to generate a plurality of sub-patterns for different subsets of features of the set of features, and further including instructions executable to apply the at least one machine learning model to the longitudinal dataset to obtain a confidence score for each of the plurality of sub-models and predict the current state of menopause based on the confidence score for each of the plurality of sub-models.

9. The non-transitory computer-readable storage medium of claim 8, wherein each of the confidence scores are weighted based on at least one of:
a level of predictiveness of the subset of features and a reliability of sensor signals associated with the subset of features.

10. The non-transitory computer-readable storage medium of claim 1, further including instructions executable to align features of the set of features to a common time point and to predict the current state of menopause for the user based on a plurality of past predicted states of menopause for the user.

11. A non-transitory computer-readable storage medium comprising instructions that when executed cause processor circuitry of a computing device to:
generate a longitudinal dataset of a set of features from tracked physical measurements of a user received from sensor circuitry;
identify a baseline pattern of the set of features for the user using at least a portion of the longitudinal dataset of the set of features;
generate at least one machine learning model based on the baseline pattern of the set of features and general population trends of a demographic population associated with the user;
identify a menopause-related anomaly in the set of features using the at least one machine learning model and based on a change from the baseline pattern of the set of features and the general population trends; and
communicate a data message indicative of the anomaly to the user.

12. The non-transitory computer-readable storage medium of claim 11, wherein the instructions to communicate the data message include instruction executable to automatically send the data message to the user indicative of an issue associated with the menopause-related anomaly and notify the user to contact a professional, and wherein the menopause-related anomaly includes an abnormal transition between menopause states.

13. The non-transitory computer-readable storage medium of claim 11, wherein the at least one machine learning model includes a first machine learning model indicative of a prediction of a current state of menopause for the user and a second machine learning model applied to generate pseudo-features from the set of features and identify the menopause-related anomaly, and further including instructions executable to predict the current state of menopause for the user using the first machine learning model that identifies the pattern within at least one of the pseudo-features and the menopause-related anomaly indicative of a probability of the user being in a state of menopause, wherein the communicated data message is indicative of the menopause-related anomaly and the current state of menopause.

14. The non-transitory computer-readable storage medium of claim 13, wherein each of the first machine learning model and the second machine learning data model include a plurality of sub-models for different groups of features of the set of features, and the instructions to apply the first machine learning model to the longitudinal data set include instructions executable to obtain a confidence score for each of the plurality of sub-models and to predict the current state of menopause based on the confidence score for each of the plurality of sub-models.

15. A system, comprising:
sensor circuitry, including communication circuitry, configured to obtain sensor signals indicative of physical measurements associated with a user and to communicate the physical measurements; and
processor circuitry configured to:
generate a longitudinal dataset of a set of features from the physical measurements of the user as tracked over time;
identify a baseline pattern of the set of features for the user identified using at least a portion of the longitudinal dataset of the set of features;
generate at least one machine learning model based on the baseline pattern of the set of features and general population trends of a demographic population associated with the user;
identify a menopause-related anomaly in the set of features using the at least one machine learning model and based on a change from the baseline pattern of the set of features and the general population trends;
identify a pattern within the set of features indicative of a probability of the user being in a state of menopause using the at least one machine learning model and at least one of the set of features and the menopause-related anomaly; and
communicate a data message indicative of the menopause-related anomaly to the user.

16. The system of claim 15, wherein the processor circuitry is configured to identify the anomaly in the set of features from the pattern using the at least one machine learning model and based on a deviation from the baseline pattern and general population trends of the demographic population associated with the user.

17. The system of claim 15, wherein the at least one machine learning model includes a first machine learning model indicative of the probability of the user being in the state of menopause and a second machine learning model that includes an encoder/decoder pair applied to generate pseudo-features from the set of features and to identify the menopause related anomaly from a reconstruction error, and the processor circuitry is configured to apply the first machine learning model to the pseudo-features to predict a current state of menopause for the user based on the identified pattern within the set of features indicative of the probability of the user being in the state of menopause, wherein the current state of menopause is associated with a transition to or from a state selected from the group consisting of:
pre-menopause, menopause transition, and post-menopause.

18. The system of claim 17, wherein the processor circuitry is configured to predict the current state of menopause for the user based on at least two or more of:
the identified pattern;
a plurality of past predicted states of menopause for the user;
the pseudo-features; and
the menopause-related anomaly.

19. The system of claim 15, further including input circuitry configured to receive information from the user, wherein the set of features include the received information.

20. The system of claim 15, wherein the processor circuitry is configured to identify the menopause-related anomaly in the set of features using the at least one machine learning model that includes a neural network data model with hidden states to identify the baseline pattern and the menopause-related anomaly is identified from the baseline pattern using the longitudinal data set, wherein the baseline pattern includes a plurality of sub-patterns associated with the set of features.
